(19) United States
(12) Patent Application Publication
(10) Pub. No.: US 2010/0042042 A1
(43) Pub. Date: Feb. 18, 2010

(54) ENVIRONMENTAL STATE DETECTION WITH HYDROGEL BASED FULLY INTEGRATED TRANSDUCER DEVICE

(75) Inventors: Ventzeslav Petrov Iordanov, Eindhoven (NL); Michel Paul Barbara Van Bruggen, Eindhoven (NL); Hendrika Cecilia Krijnsen, Eindhoven (NL); Anna-Maria Janner, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/444,722

(22) PCT Filed: Oct. 8, 2007
(86) PCT No.: PCT/IB07/54078

§ 371 Date: Apr. 8, 2009

(30) Foreign Application Priority Data
Oct. 12, 2006 (EP) 06122170.1

(51) Int. Cl. A61M 37/00; G01J 1/44; H01L 31/12; G01N 21/00

(52) U.S. Cl. 604/66; 250/206; 250/552

(57) ABSTRACT

It is described a hydrogel based transducer device for detecting an environmental state, in particular for detecting an environmental state within a biological material. The transducer device (300) comprises a base element (302), a radiation source (305), which is formed at the base element (302) and which is adapted to emit electromagnetic radiation (306), an optical element (325), which is arranged at the base element (302) and which is adapted to interact with the electromagnetic radiation (306). The transducer device (300) further comprises a radiation detector (350), which is adapted to receive the electromagnetic radiation (326) having interacted with the optical element (325), and a hydrogel material (340), which is mechanically coupled to the optical element (325) and which is adapted to change its volume when getting into contact with an environmental material of the transducer device (300) such that the spatial position of the optical element (325) is changed. The base element (302), the radiation source (305) and the radiation detector (350) are formed integrally from an electronic substrate material.

### FIELD OF INVENTION

The present invention relates to the field of detecting a physical or chemical state of a material surrounding a probe. Specifically, the present invention relates to a transducer device for detecting an environmental state, in particular for detecting an environmental state within a biological material surrounding the transducer device.

Further, the present invention relates to a medical system comprising the described transducer device.

Furthermore, the present invention relates to a method for detecting an environmental state, in particular for detecting an environmental state within a biological material, by means of a transducer device.

### ART BACKGROUND

Highly sensitive, selective, and robust sensors capable of monitoring small volumes of body fluids are one of the key components for developing responsive drug delivery systems. Protein engineering and molecular biology have facilitated the molecular design of bio-reagents, which are used as the sensing elements in various systems that offer high selectivity, good response times, and low detection limits. In addition, biosensors have been developed for physiologically relevant molecules, such as neurotransmitters and hormones.

Stimuli-sensitive hydrogels have found applications in actuators, sensors, drug delivery and bio separations. These materials are able to respond reversibly to an external stimulus that causes a distinct measurable effect on the physical properties of the material. Hydrogels are known to be sensitive to pH, ion concentration, temperature, solvent composition and electric potential. The hydrogels can be also designed to swell upon presence of a target molecule. They can be constructed in a way that the magnitude of swelling can be proportional to the concentration of ligands being present.

US 2002/0042065 A1 discloses a biosensor having a hydrogel in a rigid and preferably biocompatible enclosure. The hydrogel includes an immobilized analyte binding molecule and an immobilized analyte. The immobilized analyte competitively binds with free analyte to the analyte binding molecules, thus changing the number of cross links in the hydrogel, which changes hydrogel swelling tendency in its confined space in proportion to the concentration of free analyte concentration. By measuring the change in hydrogel pressure with a pressure transducer, the biosensor is able to accurately measure the concentration of the free analyte molecule. The described biosensor has the disadvantage that a pressure transducer is necessary, which makes a calibration of the biosensor rather difficult.

US 2004/0194523 A1 discloses a hybrid micro cantilever sensor for sensing chemical and/or biological analytes in a gaseous or liquid medium by monitoring the changes in impedance and thickness of a sensing element in the presence of the analyte. Detecting means are provided to measure the change in the physical property of the sensing material to determine the presence and/or the amount of analyte present. There is also provided an array of hybrid sensors dedicated to detecting a particular analyte, which may be included in the medium.

US 2002/056763 A1 discloses an implantable micro fabricated sensor device for measuring a physiologic parameter of interest within a patient. The sensor device includes a substrate and a sensor, integrally formed with the substrate that is responsive to the physiologic parameter of interest. At least one conductive path is integrally formed with said substrate and coupled to the sensor. Connected to the conductive path is an active circuit. The active circuit is electrically connected to the sensor.

US 2003/0100822 A1 discloses an implantable chip biosensor for in-vivo detecting an analyte in body fluids. The biosensor comprises an analyte-sensitive hydrogel slab chemically configured to vary its displacement volume according to changes in concentration of an analyte, such as glucose, in a patient's body fluid. The slab is disposed in a groove in a support block. The biosensor chip is readout by an external scanner configured to quantifiably detect changes in the displacement volume of the hydrogel slab. The support block is made of rigid or semi-rigid support material to restrain expansion of the hydrogel in all but one dimension, and the groove has one or more openings covered with a semi permeable membrane to allow contact between the patient's body fluid and the hydrogel. The scanning means may be any type of imaging devices such as an ultrasound scanner, a magnetic resonance imager, or a computerized tomography scanner capable of resolving changes in the slab's dimensions. The described implantable chip biosensor has the disadvantage that in order to operate the biosensor an external scanner is necessary.

US 2002/0155425 A1 discloses an implantable biosensor for detecting an analyte in-vivo in body fluids comprises an analyte-sensitive hydrogel filament chemically configured to vary its displacement volume according to changes in concentration of an analyte, such as glucose, in a patient's body fluid. A photometric displacement transducer placed inside the biosensor is configured to quantifiably detect changes in the displacement volume of the hydrogel filament, such as by detecting the light intensity on a photoreceptor arranged to receive light of varying intensity depending upon the displacement of the hydrogel filament. The described implantable chip biosensor has the disadvantage that several sensor components are necessary, which make (a) the manufacturing of the sensor rather complicated and (b) the structural shape of the sensor comparatively big.

There may be a need for providing a transducer device for detecting an environmental state, which transducer device can be manufactured within a compact structural shape.

### SUMMARY OF THE INVENTION

This need may be met by the subject matter according to the independent claims. Advantageous embodiments of the present invention are described by the dependent claims.

According to a first aspect of the present invention there is provided a transducer device for detecting an environmental state, in particular for detecting an environmental state within a biological material. The transducer device comprises (a) a base element, (b) a radiation source, which is formed at the base element and which is adapted to emit electromagnetic radiation, (c) an optical element, which is arranged at the base element and which is adapted to interact with the electromagnetic radiation being emitted from the radiation source and (d) a radiation detector, which is formed at the base element and which is adapted to receive the electromagnetic radiation having interacted with the optical element. The transducer device further comprises (e) a hydrogel material, which is mechanically coupled to the optical element and which is adapted to change its volume when getting into contact with an environmental material of the transducer device such that the spatial position of the optical element is changed. The base element, the radiation source and the radiation detector are formed integrally from an electronic substrate material.

This aspect of the invention is based on the idea that a fully integrated bio-sensitive transducer respectively detector device can be realized by using standard integration technology processes such as Complementary Metal Oxide Semiconductor (CMOS), bi-polar, Micro-Electro-Mechanical Systems (MEMS). This may provide the advantage that the transducer device can be manufactured very effectively in a low-cost process. Thereby, the electronic substrate material may be a wafer comprising preferably a semiconductor such as silicon or GaAs.

The electromagnetic radiation may be optical radiation in the visible range (400-700 nm). However, also other spectral ranges of radiation may be used such as for example infrared radiation or ultraviolet radiation.

The radiation source may be an optical active element such as a Light Emitting Diode (LED). However, the radiation source may also be represented by a first end of an optical waveguide, which is optically coupled to a light source such as a LED or a laser diode or a lamp. The optical waveguide may be for instance a fiber optical cable or a waveguide layer such as e.g. SiO₂.

The interaction between the optical element and the electromagnetic radiation may be of various kinds such as e.g. reflection, transmittance, absorption, shadowing, refraction, scattering, fluorescence, bioluminescence etc. Further, all kind of interactions may also change the spectral distribution of the electromagnetic radiation. By changing the spatial position of the optical element the intensity, the beam path and/or the spectral distribution of the light being received from the radiation detector may be measured.

The described hydrogel-based transducer device allows for realizing long lasting implantable sensing systems for accurately monitoring of physiological parameters outside and/or within the human or animal body. The transducer device may make use of the chemical response of the hydrogels converted to a physical change e.g. to a change in shape, light absorption, mechanic properties and/or the refractive index. This change is further converted into an electrical signal. The transducer device can contain a specific probe, which may be incorporated within the hydrogel layer or can form a complex with additional chemical/physical layers.

The volume change respectively a swelling of the hydrogel material may be based in various environmental changes such as the presence and/or the quantity of a specific molecule, being present. Further, the volume of the hydrogel material may be sensitive to chemical parameters such as the pH-value of the environment material or to physical parameters such as e.g. the temperature of the environment of the transducer device.

It has to be mentioned that the described transducer element can be adapted to measure not only an environmental state. The transducer element can be rather adapted to precisely monitor a change of the environment material. Thereby, not the absolute volume but a volume change of the hydrogel material is measured. By measuring only a difference between two volume states of the hydrogel material and not the absolute volume of the hydrogel material a calibration of the hydrogel based integrated transducer device can be carried out very easily and simultaneously the reliability of the described transducer device can be improved significantly. Since in particular in medical applications the reliability is a very important feature, the described transducer device can be used in an advantageous manner for a variety of applications.

According to an embodiment of the invention the optical element is formed integrally with the base element. This may provide the advantage that the whole transducer device can be formed as a fully integrated system. Thereby, the optical element can be formed by employing known technologies for building Micro-Electronic-Mechanical-Systems (MEMS). This means that the whole transducer device can be realized by means of low-power CMOS techniques and known MEMS techniques. In this respect MEMS techniques are all techniques wherein both (a) mechanical elements such as mechanical sensors or actuators and (b) electronic circuits are formed on one and the same electronic substrate.

According to a further embodiment of the invention the transducer device further comprises a dedicated electronic circuit arrangement for processing signals being provided by the radiation detector and/or being provided for driving the radiation source. Thereby, the dedicated electronic circuit arrangement may be formed discrete from or fully integrated with the base element, the radiation source and the radiation detector.

The dedicated electronic circuit arrangement may comprise a modulation circuit for controlling both the radiation source and the radiation detector in a modulated way in order to reduce the noise by applying known lock-in techniques. Further, the dedicated electronic circuit arrangement may comprise a microcontroller e.g. for controlling the operation of the transducer device and/or a memory for temporarily storing acquired measurement data.

According to a further embodiment of the invention the transducer device further comprises a power source, in particular a battery, for providing at least the radiation source and the radiation detector with energy. The battery may be rechargeable e.g. by means of a wireless power transmission from a corresponding battery charging device. This may provide the advantage that the battery can be charged even if the transducer is located in-vivo inside a human or animal body. In particular an inductive wireless coupling between the battery charging device and the rechargeable battery may be employed.

However, also an induced power source may be used for operating a simplified version of the described transducer system, which in this case is not equipped with a battery.

According to a further embodiment of the invention the transducer device further comprises a housing having a smooth outer surface. In this respect smooth means that the elevations within the surface are much smaller than the plane dimensions of the surface.

A smooth surface housing of the transducer device may have the advantage that in case the transducer is used in an in-vivo configuration the immune system of the human or animal body will not or at least not very quickly identify the transducer device as a foreign body. This has the effect that an encapsulation of the transducer device, by the body, will be retarded such that the expected lifetime of the transducer device within the living body will be increased significantly.

At this point it has to be mentioned that also the use of water-based materials further slows down the encapsulation process. This makes hydrogels extremely attractive as a base for an implantable long lasting chemo-physical sensor.

According to a further embodiment of the invention the transducer device further comprises a transmitter unit, which is adapted to communicate with an external receiving unit. This may provide the advantage that the transducer device can be used for monitoring a drug level within a patient's body, if the transducer device is used in an in-vivo application. Thereby, the communication with the external receiving unit is carried out in a wireless way.

Of course, the receiving unit might also be equipped with an alarm device, which is activated by the onset of a disease state such as an angina, a stroke or a recurrence of cancer. According to a modification, the transducer device is able to detect if a medicine was administered properly e.g. in the right time with the required dose. If this is not the case the transducer device can initiate a warning signal. Thereby, the bioavailability of the drug will be increased.

It has to be mentioned that the transducer device may further be provided with monitoring means. Thereby, the monitoring means can be used for a monitoring system that can sense and send data to another medical device—external to the body or implantable.

According to a further embodiment of the invention the radiation detector has a spatial resolution; in particular the radiation detector comprises an array of individual detector elements. Thereby, the array can be a linear array such that the radiation detector represents a line sensor or the array can be a two-dimensional arrangement of detector elements. A spatial resolving detector may be used in particular if the optical element changes the spatial propagation of the electromagnetic radiation being emitted from the radiation source.

According to a further embodiment of the invention the radiation detector is equipped with an anti-reflective coating. This may provide the advantage that the signal being detected by the radiation detector can be enhanced such that the signal to noise ratio of the transducer device will be increased. The anti-reflective coating may be made from a material having a lower refractive index than the semiconductor material the radiation detector is made from. This coating can be a thin film of scratch-resistant antireflection material like magnesium fluoride (MgF2), silicon dioxide (SiO2) or titanium dioxide (TiO2). For optimal performance the coating has a thickness equal to quarter of the wavelength of the used light.

The radiation detector may be realized by means of a photodiode, a PIN photodiode, a phototransistor, a photoconductor, a shottky photodiode, an avalanche photodiode or any other optical detector.

According to a further embodiment of the invention the optical element is a deflectable mirror. Using a deflectable mirror may provide the advantage that even a comparatively small volume change of the hydrogel being coupled to the deflectable mirror can cause a significant change of the spatial propagation of the electromagnetic radiation being reflected from the mirror. Therefore, a transducer device configuration being based on a deflectable mirror is in particular suitable if only a comparatively small volume change of the hydrogel is expected.

The spatial variation of the propagation direction of the radiation being reflected from the deflectable mirror can be detected preferably by means of a spatial resolving detector. However, also a radiation detector having no spatial resolution may be employed, which radiation detector is arranged such that depending on the amplitude of the deflection a more or less small fraction of the radiation intensity impinges onto the detector. Therefore, the intensity of the detected light corresponds to the degree of deflection respectively to the volume change of the hydrogel.

The deflectable mirror can be formed integrally with the base element. In this case the deflectable mirror is preferably formed by means of a MEMS procedure. The corresponding MEMS procedure may be carried out after the radiation source, the radiation detector and if applicable the dedicated electronic circuitry are formed within a wafer, preferably a semiconductor wafer made from e.g. silicon or GaAs.

In order to increase the reflectivity of the integrally formed deflectable mirror the surface of the mirror can be coated with a metal layer. In particular if the deflectable mirror is formed from silicon or poly silicon, the mirror may be coated with a metal layer, with a nitride layer, with an oxide layer and/or with any other material providing for a high reflectivity.

Alternatively, the deflectable mirror can be fixed to the base element by means of appropriate gluing techniques.

According to a further embodiment of the invention the optical element is realized by means of fluorescence molecules. Thereby, the spatial positioning of the fluorescence molecules is related to the actual volume of the hydrogel material. In this context the principle of measurement is based on the fact that when a volume change of the hydrogel occurs, the excited fluorescence molecule illuminate the radiation detector at a different solid angle, such that a different fraction of the total fluorescence light reaches the radiation detector. In this respect it is mentioned that the fluorescence light is emitted in all directions (i.e. in a solid angle of 4π).

In order to provide for an effective mechanical coupling between the hydrogel material and the optical element the fluorescence molecules can be attached to the hydrogel material by means of a layer covering at least one side of the hydrogel.

According to a further embodiment of the invention the fluorescence molecules are embedded in the hydrogel material. This may provide the advantage that the fluorescence molecules can be distributed within a comparatively big volume. Thereby, an effective excitation of the fluorescence molecules can be realized.

According to a further embodiment of the invention the radiation source is arranged relative to the radiation detector in such a manner that exclusively fluorescence light reaches a radiation sensitive side of the radiation detector. This may provide the advantage that almost no direct light being emitted from the radiation source can reach the radiation detector, causing an offset (noise) signal. Therefore, even weak fluorescence signals can be distinguished from the background signal.

According to a further embodiment of the invention (a) the radiation sensitive side comprises a recess, (b) the radiation source is located within a projection of the recess, and (c) the fluorescence molecules are located within the projection of the recess. Thereby, the direction of the projection is orientated at least angularly, preferably perpendicularly to the surface of the radiation sensitive side of the radiation detector. Such a configuration may provide the advantage that the quantum yield of the detection of fluorescence light is increased, because the radiation detector completely surrounds the fluorescence molecules. Therefore, by taking into account a small overall configuration of the transducer device, a comparatively large fraction of the generated fluorescence light will reach the radiation detector.

The radiation sensitive side of the radiation detector may have the shape of an annular ring. This means that the transducer device comprises a cylindrical symmetry. However, also other geometrical shapes of the radiation sensitive side such as a quadratic, a rectangular or any other possibly irregular shape can be employed.

According to a further embodiment of the invention the optical element is realized by means of a first optically semi reflective layer and a second optically semi reflective layer. Thereby, the two layers are oriented parallel to each other and the two layers are separated from each other by an intermediate layer comprising the hydrogel material.

The configuration described herewith comprises a Fabry Perot Resonator being formed in front of the active side of the radiation detector. Since for a given spectral distribution the intensity transmission of a Fabry Perot Resonator strongly depends on the thickness of the resonator i.e. the spacing between the two optically semi reflective. The Fabry Perot based configuration is very sensitive to even very small changes of the thickness of the hydrogel layer.

It has to be mentioned that of course the Fabry Perot Resonator can also be used in connection with a radiation detector having a spectral resolution. Thereby, the spectral distribution of the light being transmitted through the resonator reflects the actual thickness of the hydrogel layer.

Further it has to be mentioned that the spectral resolution respectively the sensitivity of the Fabry Perot Resonator depends on the reflectance respectively the transmittance of the semi reflective layers. The bigger the transmittance is, the bigger is the spectral resolution respectively the sensitivity of the transducer device.

The radiation sensitive side of the radiation detector being equipped with a Fabry Perot Resonator may have the shape of a circle or an annular ring. This means that the transducer device comprises a cylindrical symmetry. However, also other geometrical shapes of the radiation sensitive side such as a quadratic, a rectangular or any other possibly irregular shape may be used.

According to a further embodiment of the invention the first optically semi reflective layer is formed on a radiation sensitive side of the radiation detector. This may provide the advantage that the Fabry Perot Resonator is located directly onto the radiation detector. Therefore, the whole transducer device can be realized within a comparatively small and compact design.

According to a further embodiment of the invention the optical element is a shadowing element, which is located at least partially within the electromagnetic radiation path extending from the radiation source to the radiation detector. Thereby, the shadowing element is coupled to the hydrogel material in such a manner that the fraction of electromagnetic radiation reaching the detector strongly depends on the volume of the hydrogel material. Depending on the special design of the hydrogel material this may allow for a quick and precise detection of the environmental state.

It has to be mentioned that the fraction of electromagnetic radiation reaching the radiation detector can be measured by means of an integrating detector, which measures simply the intensity of the electromagnetic radiation. However, also a spatial resolving detector may be used in order to precisely measure the radiation intensity impinging onto the radiation detector.

According to a further embodiment of the invention the shadowing element is arranged on a radiation sensitive side of the radiation detector. This may provide the advantage that the whole hydrogel based transducer device can be realized within a comparatively small and compact design.

According to a further embodiment of the invention the shadowing element is a movable mirror. This may provide the advantage that an effective shadowing element can be realized by means of a comparatively thin layer of an appropriate reflecting material. A reflection based shadowing may further provide the advantage that there is no or only little radiation absorption. Therefore, the shadowing element will generate no of only a negligible temperature rising of the transducer device even if the shadowing element blocks all radiation from the radiation detector.

According to a further aspect of the invention there is provided a medical system. The provided medical system comprises (a) the transducer device according any one of the embodiments described above and (b) a drug release device, which is coupled to the transducer system and which is adapted to release a certain amount of drug when being triggered by the transducer system.

This aspect of the invention is based on the idea that an automatic medication application can be realized by coupling the above-described transducer device with an appropriate drug release device. Thereby, the transducer device may be incorporated in-vivo within a patient's body. Upon detection of a predefined environmental state or an environmental change of the transducer device a drug release can be triggered. The medicated drug dose may be related to the environmental state or to the strength of the environmental change. In other words when the transducer device senses targeted molecules or an environmental change, the transducer device produces an electrical signal, which can trigger drug release e.g. from a reservoir incorporated in the medical system.

It has to be mentioned that of course the electrical signal of the temporal progression of the electrical signal can also be stored and later accessed by a physician. An advanced transducer device may also send data through wireless communication link to another in-body system or outside of the body.

In the following, an advantageous exemplary application of the medical system will be briefly described: Sometimes patients suffering from myocardial infarction cannot reach the emergency room in time; they would receive valuable, life-sustaining benefit from implanted sensors for cardiac markers with the described medical system representing a closed-loop drug delivery system.

An additional advantage of the implantable responsive medical system is that it may continuously monitor a set of parameters and disease markers in patients with known risk factors. Physicians could closely follow the changes in the patient's health by examining the data obtained by the sensing device.

According to a further aspect of the invention there is provided a method for detecting an environmental state, in particular for detecting an environmental state within a biological material, by means of a transducer device. The provided method comprises the steps of (a) emitting electromagnetic radiation form a radiation source, which is formed at a base element of the transducer device and (b) directing the electromagnetic radiation to an optical element, which is arranged at the base element. Thereby, the optical element is coupled to a hydrogel material, which is adapted to change its volume when getting into contact with an environmental material of the transducer device such that the spatial position of the optical element is changed. The provided method further comprises (c) receiving the electromagnetic radiation, which has at least partially interacted with the electromagnetic radiation being emitted from the radiation source, by means of a radiation detector. The base element, the radiation source and the radiation detector are formed integrally from an electronic substrate material.

This aspect of the invention is based on the idea that the state of an environmental material can be measured by means of a fully integrated bio-sensitive transducer respectively detector device. This may provide the advantage that the transducer device can be manufactured very effectively e.g. by employing known and standard Integrated Circuits (IC) technologies. Thereby, the electronic substrate material may be a wafer comprising preferably a semiconductor.

The electromagnetic radiation may be in particular optical radiation in the visible part of the spectrum. However, also other spectral ranges of radiation may be used such as for example infrared radiation or ultraviolet radiation.

Interaction between the optical element and the electromagnetic radiation may be of various kinds such as e.g. reflection, transmittance, absorption, shadowing, refraction, fluorescence, bioluminescence etc. Further, all kind of interactions may also change the spectral distribution of the electromagnetic radiation. By changing the spatial position of the optical element the intensity, the beam path and/or the spectral distribution of the light being received from the radiation detector is measured.

The volume change of the hydrogel material may be based in various environmental changes such as the presence and/or the quantity of a specific molecule. Further, the described method can be applied both in-vivo and in-vitro.

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG. 1 shows a schematic illustration of a transducer device, which can be used as a drug monitoring device.

FIG. 2 shows a medical system comprising a transducer device and a drug release device being coupled to the transducer device by means of a wireless transmission route.

FIG. 3a shows a cross sectional view of a hydrogel based transducer device comprising a deflectable mirror.

FIG. 3b shows a drawing indicating the geometry of light paths, which develop in the hydrogel based transducer device as depicted in FIG. 3a.

FIG. 3c shows deflectable mirrors, which are formed on a substrate by using MEMS techniques.

FIG. 4a shows a cross sectional view of a hydrogel based transducer device comprising fluorescence molecules being embedded in a hydrogel layer.

FIG. 4b shows a top view of the hydrogel based transducer device as depicted in FIG. 4a.

FIG. 4c shows a drawing for calculating solid angles of fluorescence radiation reaching the detector of the hydrogel based transducer device as depicted in FIG. 4a.

FIG. 5a shows a cross sectional view of a hydrogel based transducer device comprising a Fabry Perot Resonator being formed at opposite surfaces of a hydrogel layer.

FIG. 5b shows a top view of the hydrogel based transducer device as depicted in FIG. 5a.

FIG. 6 shows a cross sectional view of a hydrogel based transducer device comprising a movable mirror element for shadowing at least a portion of radiation being directed to an integrally formed radiation detector.

FIG. 7 shows a drawing illustrating a swelling respectively a deswelling of a hydrogel material upon a change in concentration of a monitored analyte.

### DETAILED DESCRIPTION

The illustration in the drawing is schematically. It is noted that in different figures, similar or identical elements are provided with the same reference signs or with reference signs, which are different from the corresponding reference signs only within the first digit.

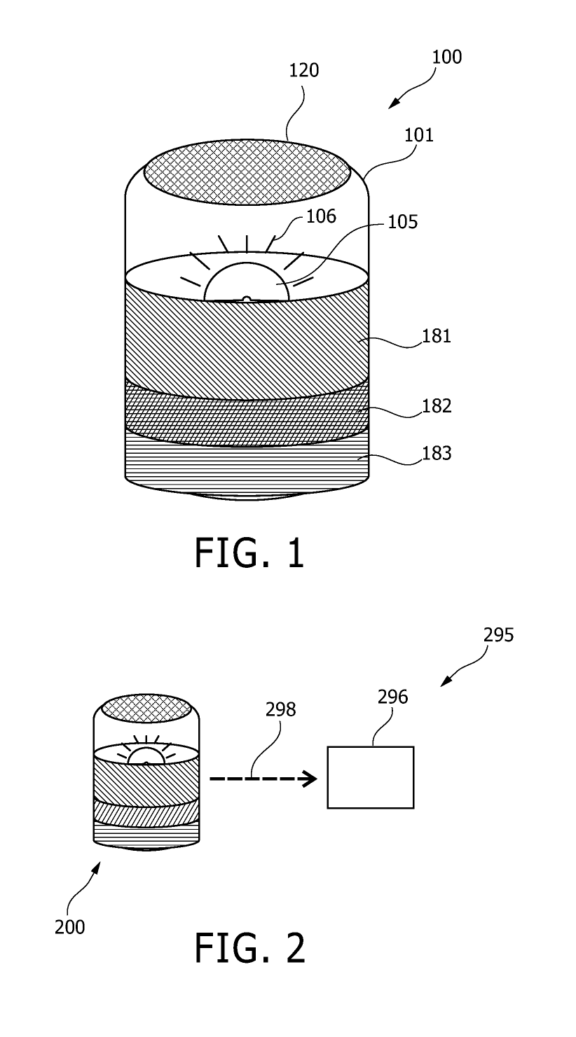

FIG. 1 shows a simple schematic diagram of a transducer device 100, which can be used as a drug-monitoring device. The transducer device 100 comprises a housing 101, in which a plurality of components of the transducer device 100 are embedded. The outer surface of the housing 101 is smooth such that in an in-vivo application of the transducer device 100 an encapsulation of the transducer device 100 is decelerated. Such an encapsulation is typically caused by the immune system of the human or animal body, which will sooner or later identify the transducer device 100 as a foreign body. Due to a retarded encapsulation of the transducer device 100 the life-time of the transducer device 100 within a patient's body will be reduced.

The transducer device 100 comprises a radiation source 105, which according to the embodiment described here is a light emitting diode 105. The light emitting diode 105 is formed integrally with an electronic substrate, which is not depicted in FIG. 1. The light emitting diode 105 emits electromagnetic radiation 106, which reaches a sensor block 120. The sensor block 120 is formed with the electronic substrate respectively the light emitting diode 105 in an at least partially integrally manner. The components and different embodiments of the sensor block 120 will be described below in detail.

The transducer device 100 further comprises dedicated electronics 181, which include electronic circuit arrangements for driving the light emitting diode 105 and/or for data evaluating of signals being provided by a not depicted radiation detector. The electronic circuit arrangement comprises a modulation circuit for controlling both the radiation source and the radiation detector in a modulated way in order to reduce the noise by exploiting known lock-in techniques. The dedicated electronics 181 also comprises a microcontroller e.g. for controlling the operation of the transducer device and/or the operation of a memory for temporarily storing acquired measurement data.

Further, the transducer device 100 comprises a power source 182, which according to the embodiment described here is a battery 182. The battery 182 may be rechargeable e.g. by means of a wireless power transmission from a corresponding battery charging device.

Furthermore, the transducer device 100 is equipped with a transmitter unit and/or receiver unit 183. The transmitter unit is adapted to communicate with a not depicted external receiving unit. Therefore, if the transducer device is used in an in-vivo application, the transducer device 110 can be used for monitoring a drug level within a patient's body. Thereby, the communication with the external receiving unit is carried out in a wireless manner.

FIG. 2 shows a medical system 295, which comprises a transducer device 200. The transducer device 200 corresponds to the transducer device 100 illustrated in FIG. 1. The medical system 295 further comprises a drug release device 296, which is coupled to the transducer device 200 by means of a wireless transmission route 298. The drug release device 296 is equipped with a reservoir being not depicted, which reservoir is adapted for receiving a medicament. Upon detection of a predefined environmental state or an environmental change of the transducer device 200 a drug release can be triggered. The medicated drug dose can be related to the environmental state or to the strength of the environmental change. In other words, when the transducer device 200 senses targeted molecules or an environmental change, the transducer device 200 produces an electrical signal, which can trigger drug release e.g. from the reservoir being incorporated in the medical system 295.

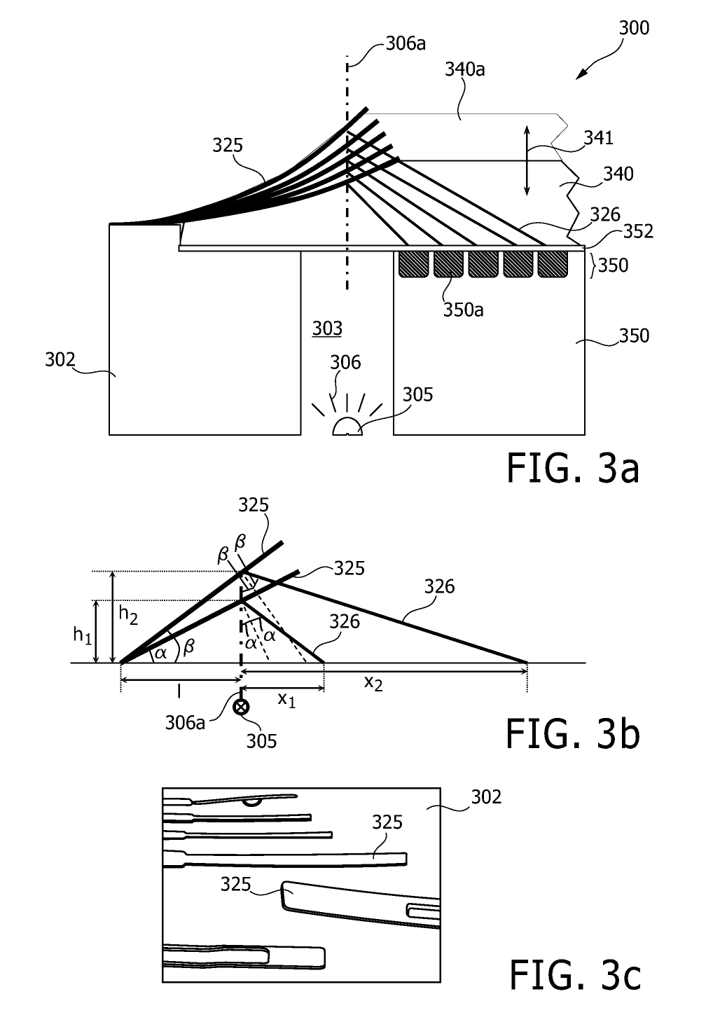

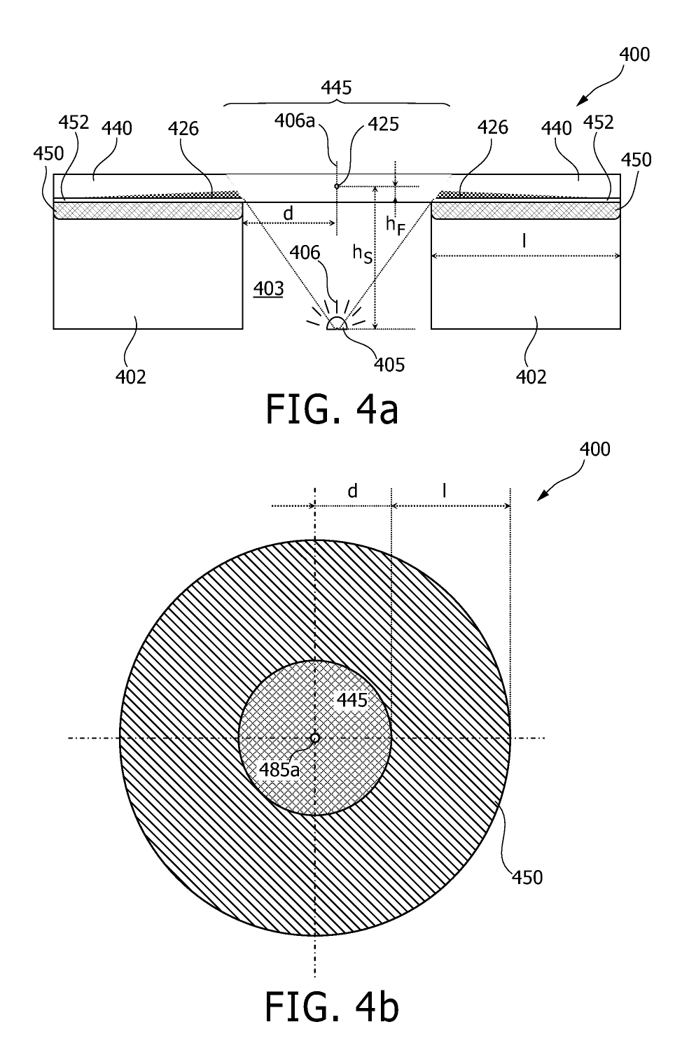

FIG. 3a shows a cross sectional view of a hydrogel based transducer device 300. The transducer device 300 comprises a base element 302, which is made from an electronic substrate. Within the electronic substrate 302 there is formed a recess 303, which accommodates a light emitting diode 305. The light emitting diode 305 emits electromagnetic radiation 306 along an optical axis 306a in an upward direction.

On a top surface of the electronic substrate 302 there is formed an optical element 325, which is adapted to interact with the electromagnetic radiation 306. According to the embodiment described here, the optical element 325 is a deflectable mirror. The deflectable mirror 325 is formed integrally from the substrate 302 by applying an appropriate MEMS technique. Since the electronic circuitry of the transducer device 300 is formed by using standard, e.g. CMOS, techniques, the whole transducer device 300 is realized with a fully integrated design by applying first the CMOS techniques and later on appropriate MEMS techniques.

The deflectable mirror 325 is mechanically coupled to a hydrogel material 340. The hydrogel material 340 is located on an anti reflective coating 352, which is a thin film transparent layer comprising for instance an oxide or a nitride layer. The thin film transparent layer 352 is formed on the top surface of the substrate 302 respectively on an active surface of a radiation detector 350.

The radiation detector 350 comprises a plurality of individual detector elements 350a, which are arranged in a linear array. Therefore, the radiation detector 350 allows for a spatial resolution, whereby the individual detector elements 350a are appropriately coupled to the electronic circuitry of the transducer device 300. The electromagnetic radiation 306 being emitted from the radiation source 305 is reflected at the optical element 325 such that electromagnetic radiation 326 impinges onto the radiation detector 350.

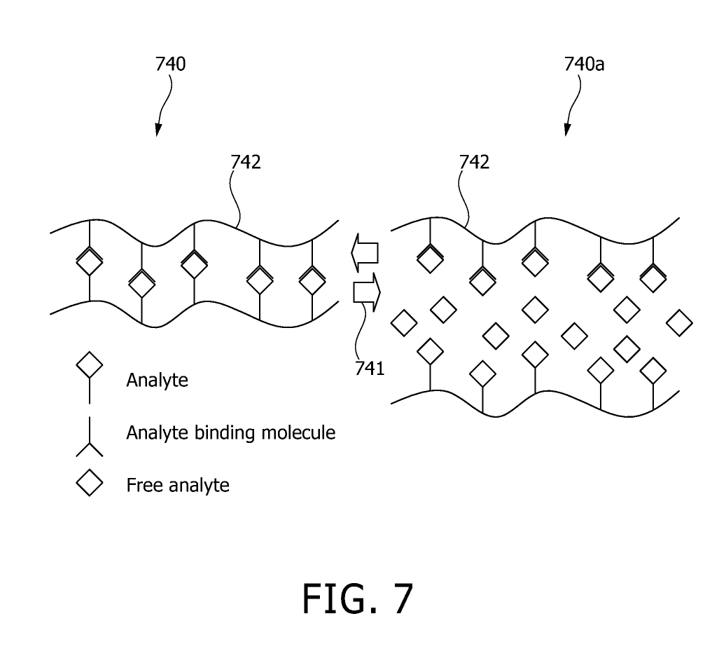

The hydrogel material 340 is accommodated in a form, which for sake of clarity is not depicted in FIG. 3a. The form is shaped in such a manner, that when operating the transducer device 300 the hydrogel material 340 comes into contact with the environmental material surrounding the transducer device 300, which environmental material comprises for instance a certain concentration of bio-molecules. When this concentration changes, the hydrogel material 340 will change its volume. Thereby, as indicated by the arrow 341, the hydrogel material 340 will swell such that the hydrogel material will assume an expanded state 340a. This expansion of the hydrogel material 340 will cause a further deflection of the deflectable mirror 325. Therefore, the further mirror deflection represents an environmental change of the material with which the hydrogel 340 is in contact with respectively of the material surrounding the transducer device 300. The chemical principle which responsible for the volume change of the hydrogel 340 will be explained in more detail below with reference to FIG. 7.

Upon swelling of the hydrogel material 340, 340a, the reflected electromagnetic radiation 326 will impinge onto the spatial resolving detector 350 at a different position. This positional variation is a measure for the strength of the environmental change.

At this point it is emphasized that if the radiation source is e.g. a light emitting diode 305 transmitting a non collimated radiation 306, of course the electromagnetic radiation 326 will impinge onto the detector with a comparatively wide spatial distribution. However, upon a further bending of the deflectable mirror 325, the position of the spatial center of this spatial distribution will move. The positional shift of this spatial distribution can be detected easily with the radiation detector 350.

It is further pointed out that this configuration of the transducer device 300, where the deflectable mirror 325 bends upon the hydrogel swelling, can also be realized with a spatially integrating detector having no spatial resolution. In this case a further bending of the deflectable mirror 325 will have the effect, that not all light being reflected by the mirror 325 will reach the detector 350. Therefore, the intensity of the measured light 326 will vary depending on the hydrogel swelling respectively the environmental state of the material surrounding the transducer device 300.

FIG. 3b shows a drawing indicating the trigonometry of the light paths, which develop in the hydrogel based transducer device 300. Thereby, there is made the assumption that the deflectable mirror 325 is a straight structure, which depending on the strength of the hydrogel swelling adopts a different angular position α respectively β with respect to the surface of the substrate 302. Further, there is made the assumption that the light source 306 emits a collimated non-diverging beam along the optical axis 306a. With the help of FIG. 3b, one can derive easily the following equations:

$$\begin{matrix}tan α = \frac{{h}_{1}}{l} and tan β = \frac{{h}_{2}}{l} & [ 1 ] \\ tan 2 α = \frac{{x}_{1}}{{h}_{1}} and tan 2 β = \frac{{x}_{2}}{{h}_{2}} & [ 2 ]\end{matrix}$$

Thereby, h₁ and h₂ are the height positions, where the radiation beam hits the deflectable mirror 325. The parameter l is the horizontal distance between optical axis 306a and the position where the straight mirror 325 is fixed to the substrate 302 in a swiveling manner. The angles 2α and 2β are the reflection angles of the light paths 326 with respect to the surface of the straight deflectable mirror 325.

Using the universal valid trigonometric formulas

$$tan 2 α = \frac{2 tan α}{1 - {tan}^{2} α} and tan 2 β = \frac{2 tan β}{1 - {tan}^{2} β} \qquad [ 3 ]$$

one obtains the following expression:

$$\frac{{x}_{2}}{{x}_{1}} = \frac{{lh}_{2}^{2} ( 1 - {tan}^{2} α )}{{h}_{1} tan α ( {l}^{2} - {h}_{2}^{2} )} = \frac{{( \frac{{h}_{2}}{{h}_{1}} )}^{2}}{( {( \frac{l}{{h}_{1}} )}^{2} - {( \frac{{h}_{2}}{{h}_{1}} )}^{2} )} \frac{( 1 - {tan}^{2} α )}{\frac{{h}_{1}}{l} tan α} \qquad [ 4 ]$$

Thereby, x₁ and x₂ are the positions where the reflected light 326 impinges onto the detector 350.

When defining the hydrogel expansion factor

$$κ = \frac{{h}_{2}}{{h}_{1}} ,$$

one can get:

$$\frac{{x}_{2}}{{x}_{1}} = \frac{{cot}^{2} α - 1}{\frac{{cot}^{2} α}{{κ}^{2}} - 1} = \frac{{κ}^{2} ( 1 - {tan}^{2} α )}{1 - {κ}^{2} {tan}^{2} α} \qquad [ 6 ]$$

The initial distance to the detector array can be derived from:

$${x}_{1} = \frac{2 l}{{cot}^{2} α - 1} , \qquad [ 7 ]$$

Now considering typical application parameters values: l=100 μm, α=30° and κ=1.03 corresponding to a 3% increase in lateral size respectively a 10% increase in volume, one can easily calculate:

x₁=100 μm and x₂=109.4 μm, [8]

As can be seen from the results even a small hydrogel volume change upon swelling will introduce significant change in the position of the reflected light spot onto the detector 350. This makes this configuration of the transducer device 300 also suitable for hydrogels 340 that undergo small volume change only. Increasing the initial angle of incidence α the difference in the distances of the center of the reflected spot from the center of the entering light increases significantly. However, one has to consider that of course the angle of incidence should never exceed 45° degree, because otherwise the reflected radiation 326 will not reach the detector 350 being arranged below the deflectable mirror 325.

The hydrogel can be also fixed to the substrate at one side, leading to lateral change in only one direction. Then 10% change in volume will result in 10% lateral change, making the system more sensitive (e.g. suitable for extremely small changes).

FIG. 3c shows an electronic substrate 302, onto which deflectable mirrors 325 are integrally formed. The deflectable mirrors 325 have the shape of cantilevers. Preferably, the deflectable mirrors 325 are formed by using known MEMS techniques. When manufacturing the transducer device 300 in a fully integrated configuration, in a first standard CMOS procedure performed on a silicon wafer the recess 303, the light source 305, the detector 350 and a not depicted electronic circuitry are formed. In a second post process the thin film transparent layer 352 and the deflectable mirrors 325 are formed.

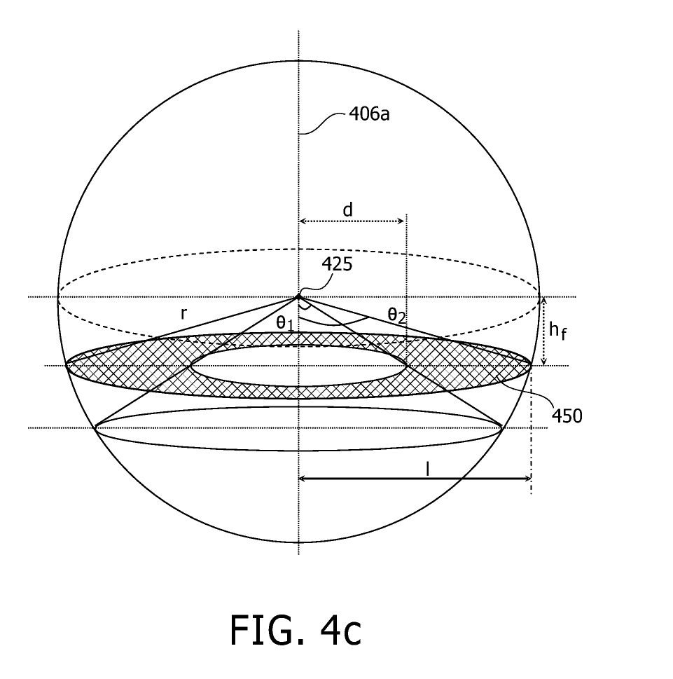

FIG. 4a shows a cross sectional view of a hydrogel based transducer device 400. The transducer device 400 comprises a base element 402, which is made from an electronic substrate. Within the electronic substrate 402 there is formed a recess 403, which accommodates a light emitting diode 405. The light emitting diode 405 emits electromagnetic radiation 406 along an optical axis 406a in an upward direction.

On a top surface of the electronic substrate 402 there is formed a radiation detector 450. According to the embodiment described here the electronic substrate 402 respectively the radiation detector 450 comprises a cylindrical symmetry with the recess respectively the light source 405 being arranged in the center. Both the radiation source 405 and the radiation detector 450 may be formed integrally with the electronic substrate 402 by applying known standard CMOS technologies for manufacturing electronic and optoelectronic circuitries.

As can be seen from FIG. 4a, a thin film transparent layer 452 is formed on the top surface of the substrate 402 respectively on an active surface of a radiation detector 450. On the layer 452 there is arranged a hydrogel material 440. Within the hydrogel material 440 there are embedded fluorescence molecules 425, which represent an optical element. The fluorescence molecules 425 are embedded at least within a region of excitation 445 of the hydrogel layer 440, which region 445 is located above the recess 403.

Light 406 being emitted from the radiation source 405 excites the fluorescence molecules 425. After a deexcitation of the fluorescence molecules 425 fluorescence light 426 will be emitted into a full solid angle of 4π. This means that the fluorescence light 426 will be transmitted in all directions. However, a certain fraction of the will impinge onto the light sensitive upper side of the radiation detector 450. Thereby, the solid angle of radiation 426 reaching the detector 450 depends on the vertical position of the fluorescence molecules 425 with respect to the light sensitive surface of the radiation detector 450.

When operating the transducer device 400 for sensing environmental changes, the hydrogel material 440 comes into contact with environmental material surrounding the transducer device. Thereby, a swelling as described above in connection with FIG. 3a occurs. It is clear that a swelling of the hydrogel material 440 will cause a vertical positional shift of the embedded fluorescence molecules 425 such that the fraction of detected fluorescence radiation 426 will change.

The hydrogel material 440 is accommodated in a form not depicted in FIG. 4a. The form is shaped in such a manner, that hydrogel material 440 may come into contact with the environmental material of the transducer device 400, the vertical dimension i.e. the thickness of the hydrogel material 440 changes. However, it has to be pointed out that also a horizontal shift of the hydrogel material 440 upon coming into contact with a changed environment may be possible to detect environmental changes. Thereby, it is necessary that the fluorescence molecules 425 are predominately embedded within the region 445 and are not homogeneously distributed over the whole hydrogel material 440.

FIG. 4b shows a top view of the hydrogel based transducer device 400. The light sensitive surface of the radiation detector 450 having an annular shape formed symmetrically around the optical axis 406a can be seen. In the center of the radiation detector 450 there is formed the excitation region 445. The excitation region 445 has a radius d, the radiation detector 450 has a wall thickness l leading to radius d+l of the radiation source 450.

It has to be mentioned that of course also other geometrical shapes of the radiation detector 450 respectively the whole transducer device 400 may be possible. Such shapes include for instance a quadratic, a rectangular, an elliptic or any other irregular shape.

In the following a calculation presenting an estimation of the sensitivity of such a fluorescence based transducer device 400 will be given. Thereby, reference is made to FIG. 4c showing a drawing for calculating solid angles of fluorescence radiation 426 reaching the detector 450 of the hydrogel based transducer device 400 as depicted in FIG. 4a.

Considering that the fluorescence molecules are homogeneously distributed, the following calculations are made under the assumption that all of the fluorescence molecules are concentrated in the geometrical center of the irradiated region volume 445. It has turned out that this assumption does not cause significant deviations from the real situation. The fraction from the fluorescence light reaching the detector can be expressed with:

$$\frac{{I}_{F}}{{I}_{TF}} = \frac{{Ω}_{F}}{{Ω}_{TF}} \qquad [ 9 ]$$

Thereby, I_{F} is the intensity of the fluorescence light reaching the detector, I_{TF} is the total fluorescence radiation, Ω_{F} is the segment of the total solid angle corresponding to the detectable fluorescence light and, Ω_{TF}=4π is the total solid angle. Ω_{F} can be calculated using FIG. 4c and following the equations:

$$\begin{matrix}\begin{matrix}d Ω = \frac{dA}{{r}^{2}} \\ = \frac{r d Θ r sin Θ d α}{{r}^{2}} \\ = sin Θ d Θ d α ,\end{matrix} & [ 10 ] \\ \begin{matrix}{Ω}_{F} = {\int }_{0}^{2 π} d α {\int }_{{Θ}_{1}}^{{Θ}_{2}} sin Θ d Θ \\ = 2 π {\vert - cos Θ \vert}_{{Θ}_{1}}^{{Θ}_{2}} = \\ = 2 π ( cos {Θ}_{1} - cos {Θ}_{2} ) \\ = 2 π ( \frac{1}{\sqrt{1 + {( \frac{d}{{h}_{F}} )}^{2}}} - \frac{1}{\sqrt{1 + {( \frac{l}{{h}_{F}} )}^{2}}} )\end{matrix} & [ 11 ]\end{matrix}$$

If a thin-film hydrogel layer is used, the following terms hold:

$${( \frac{d}{{h}_{F}} )}^{2} >> 1 and ( \frac{l}{{h}_{F}} ) >> 1 \qquad [ 12 ]$$

Therefrom, one obtains the following expression:

$${Ω}_{F} = 2 π {h}_{F} ( \frac{1}{d} - \frac{1}{l} ) \qquad [ 13 ]$$

Now considering typical application parameters values: d=100 μm, l=200 μm and h_{F}=5 μm one can easily calculate:

$$\begin{matrix}\frac{{I}_{F}}{{I}_{TF}} = \frac{{Ω}_{F}}{{Ω}_{TF}} \\ = \frac{{h}_{F}}{2} ( \frac{1}{d} - \frac{1}{l} ) \\ = \frac{{h}_{F}}{2 d} \\ = \frac{{h}_{F}}{l} \\ = 0.025\end{matrix} \qquad [ 14 ]$$

This means in other words that 2.5% of the total emitted fluorescence radiation will be collected by the radiation detector 450.

In the following an estimation of a typically expected electrical signal produced by the fluorescence based transducer device 400 will be given:

The fluorescence is mainly affected by the following parameters:

a) The excitation photon flux reaching incident energy Φₑₓ [photons s⁻¹ sr⁻¹]:

$${Φ}_{ex} = {E}_{ex} \frac{λ}{hc} , [ ph {s}^{- 1} ] \qquad [ 15 ]$$

Thereby, Eₑₓ is the optical power of the excitation beam in the unit Watt W.

b) The fluorescence energy yield q is the ratio of fluorescence energy emitted to the energy absorbed. This parameter is material dependent. Good flourophores have an energy yield ratio greater than 1/2.
c) The solid angle of collection Ω_{F} i.e. the total angle over which emitted light is collected. This is a parameter, which can be easily varied in the detector design to further improve the sensitivity.
d) The extinction coefficient ε_{κ} of the excitation light in the hydrogel material is the variable most commonly optimized. Modifying the chemical composition of the material can alter the extinction coefficient.
e) The fluorescence signal is proportional to the light path length lₚ in the solution. To maximize absorption the incident light has to pass through as much as possible of the irradiated compound.
f) The fluorescence signal will increase linearly with the concentration K as long as the sample is within the linear dynamic range.
The entire equation for the fluorescence signal is then defined by:

$${S}_{f} \approx {E}_{ex} \frac{λ}{hc} η \frac{{Ω}_{F}}{{Ω}_{TF}} {ɛ}_{κ} {l}_{p} κ \qquad [ 16 ]$$

The unit of S_{f} is [photons s^{=1].}

An example for a fluorescence molecule 425 that can be used in the transducer device 400 is ATTO 520, which can be obtained from ATTO-TEC GmbH, P.O. Box 10 08 64, D-57008 Siegen; Germany. Physical properties of ATTO 520 are given in the following table 1:

TABLE 1
Physical properties of the fluorescence molecule ATTO 520

| Symbol | Description | Value | Unit |
|---|---|---|---|
| λ_{abs} (peak) | The longest wavelength | 525 | [nm] |
| | absorption maximum | | |
| ε_{κ} | Extinction coefficient | 1.1 × 10⁵ | [Mol⁻¹ cm⁻¹] |
| λₑₘ (peak) | Fluorescence emission maximum | 545 | [nm] |
| η | Quantum yield | 90 | [%] |

Having free fluorescence molecules 425 with concentration 1 μMol, optical path length of 0.005 mm and an optical source 405 with luminous intensity of about 250 mcd, one can make estimation for the produced amount of fluorescence photons per second. The result is given in the following table 2.

TABLE 2
Parameters for estimating a required amount
of fluorescence photons per second

| Symbol | Description | Value | Unit |
|---|---|---|---|
| h | Planck's constant | 6.6 × 10⁻³⁴ | [J s] |
| c | Speed of light | 3 × 10¹⁰ | [cm s⁻¹] |
| λ_{abs} | Absorption maximum | 525 × 10⁻⁷ | [cm] |
| ε_{κ} | Extinction coefficient | 1.1 × 10⁵ | [M⁻¹ cm⁻¹] |
| η | Quantum yield | 90 | [%] |
| Ω_{F}/Ω_{TF} | Solid angle portion | 2.5 | [%] |
| Eₑₓ | Luminous intensity | 200 mcd ≈ 300 μW | [W] = [J s⁻¹] |
| lₚ | Light path | 5 × 10⁻⁴ | [cm] |
| κ | Molecule | ~1 × 10⁻⁶ | [M] |
| | concentration | | |
| S_{f} | Fluorescence signal | ~10⁹ | [photons s⁻¹] |

Assuming that the photo detector 450 has response R of 0.35 AW⁻¹ at the wavelength of 545 nm one can easily estimate the amount of the electrical signal. Thereby, one finally obtains the following result for the estimated current produced by the transducer device 400:

$$I = {S}_{f} \frac{hc}{λ} R = 127 \times {10}^{- 12} A \qquad [ 17 ]$$

Thereby, the unit A stands for ampere.

The transducer device 400 can be realized by using photodiodes, PIN photodiodes, phototransistors, photoconductors, Schottky photodiodes or avalanche photodiodes as a radiation sensitive optical detector 450. In order to increase the overall signal one can carry out a signal integration for longer time intervals e.g. extending 1 s. The signal can further be enhanced by placing an appropriate thin film anti-reflective coating 452 above the detector 450. For the coating 452 a material having a higher refractive index as compared to the material of the detector 450 may be used. Such a material is e.g. silicon oxide or silicon nitride.

The transducer device 400 shown in FIG. 4a can be implemented using a standard process technology such as e.g. CMOS plus some ‘in-’ and/or ‘post-’ process steps. The detector 450 and the required electronic circuitry can be implemented initially into a silicon wafer. The post process steps are for example involved in the formation of the recess 403 for the excitation light source 405. The layer 452 representing a light window can be created using MEMS techniques such as e.g. dry and wet etching. The hydrogel material 440 is deposited and polymerized in a last process step.

The fluorescence based transducer sensor 400 is especially suitable for hydrogels 440 that significantly change in volume upon swelling. Typical values for swelling are 100%, i.e. the volume of the hydrogel material 440 may change with a factor of two. An example for such a gel is methacrylic acid (MMA). This hydrogel swells more than 15 times (in volume) upon change in the pH of the surrounding environment in the range between ph=4 and ph=9. This leads to lateral change of about 2.5 times in the distance h_{F} indicated in FIG. 4a.

It has to be mentioned that a non depicted form may be provided for the hydrogel material 440, which form limits the structural swelling of the hydrogel 440 to the vertical z-direction. This will further enhance the change in the signal provided by the fluorescence molecule based transducer device 400.

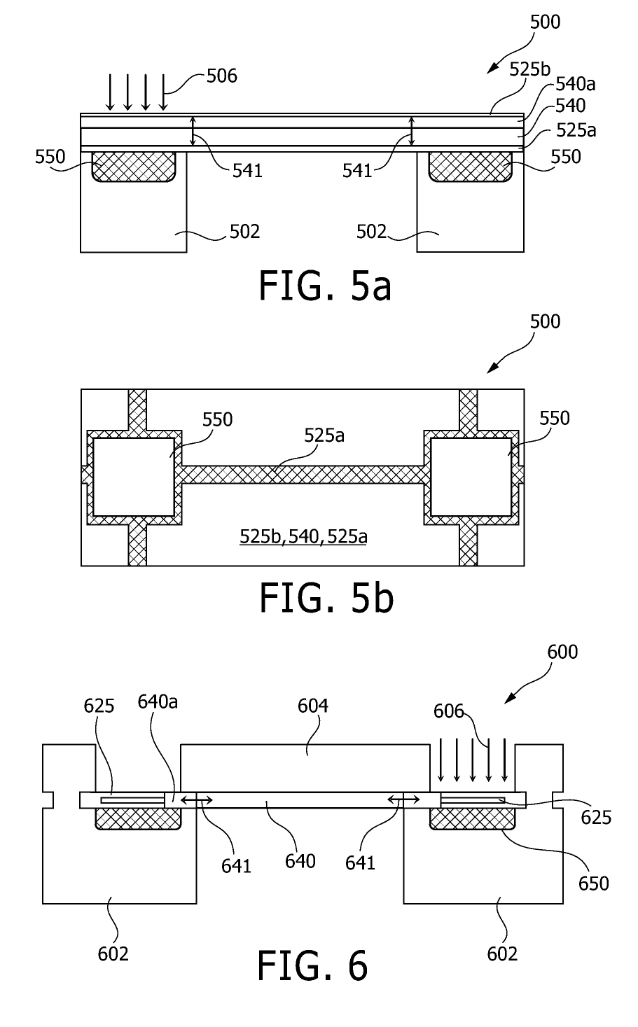

FIG. 5a shows a cross sectional view of a hydrogel based transducer device 500. The transducer device 500 comprises a base element 502, which is made from an electronic substrate.

On a top surface of the electronic substrate 502 there is formed a radiation detector 550. According to the embodiment described here the electronic substrate 502 respectively the radiation detector 550 comprises a cylindrical symmetry. The radiation detector 550 may be formed integrally with the electronic substrate 502 by applying known standard CMOS technologies for manufacturing electronic and optoelectronic circuitries.

On the upper surface of the electronic substrate 502 respectively the radiation detector 550 there is provided a Fabry Perot Resonator comprising first semi reflective layer 525a and a second semi reflective layer 525b. In between the two semi reflective layers, which in combination represent an optical element of the transducer device 500, there is formed a hydrogel layer 540. In other words, the hydrogel layer 540 is sandwiched by the first semi reflective layer 525a and a second semi reflective layer 525b.

When the hydrogel material 540 comes into contact with an environmental change of the transducer device 500, a swelling of the hydrogel 540 will occur leading to an expanded state 540a. This swelling along the vertical direction is indicated by the arrow 541.

According to the known principles of a Fabry Perot Interferometer electromagnetic radiation 506, which is emitted by a not depicted light source, impinges onto the second semi reflective layer 525b. Due to the semi reflectivity of the two layers 525a and 525b an optical resonator is formed on top of the radiation detector 550. The length of this resonator strongly depends on the thickness of the hydrogel layer 540, which thickness itself depends on the environmental state of the material surrounding the transducer device 500. The resonator thickness has a strong impact on the spectral distribution and as a consequence also on the radiation intensity reaching the detector 550 being positioned below the layer 525a. Therefore, by measuring the light intensity and/or the spectral distribution of the radiation reaching the detector 550, the resonator thickness and as a consequence the environmental state of the transducer device 500 can be evaluated. Of course, the signal evaluation strongly depends on the spectral distribution of the incident light 506.

FIG. 5b shows a top view of the Fabry Perot based transducer device 500. On top there can be seen the Fabry Perot resonator comprising the second semi reflective layer 525b, the hydrogel material 540 and the first semi reflective layer 525a. The first semi reflective layer 525a comprises a structured shape covering predominately the radiation sensitive surfaces of the radiation detector 550. The radiation detector 550 comprises a plurality of different detector elements being shifted laterally with respect to each other. A plurality of these detector elements may be arranged in a one-dimensional or in a two-dimensional array.

FIG. 6 shows a cross sectional view of a hydrogel based transducer device 600. The transducer device 600 comprises a first base element 602 and two radiation detectors 650 being formed integrally with the first base element 602. The first base element is an electronic substrate 602. The transducer device 600 further comprises a second base element 604, which may also be made from an electronic substrate. In between the two base elements 602 and 604 there is provided a hydrogel material 640. The two base elements 602 and 604 represent a form allowing only for a lateral expansion of the hydrogel material 640. Therefore, when the hydrogel material 640 comes into contact with a changed environmental material surrounding the transducer device 600, the hydrogel material 640 will expand horizontally leading to an expanded state 640a. The expansion is indicated by the arrow 641.

The hydrogel material 640 is mechanically coupled with two shadowing devices 625, which at least partially block an incident light 606, which is emitted from a non depicted radiation source, form reaching the detectors 650. When the hydrogel material 640 expands or contracts horizontally, the fraction of the incident light 606 reaching the detectors 650 will vary. Therefore, the received light sensitivity is a measure for the state of the environmental material surrounding the transducer device 600.

The shadowing element 650, which in terms of the transducer device described in this application represents the optical element, is preferably a moveable mirror. A light reflection caused by the mirror has the advantage that there is no or only negligible light absorption such that the temperature of the mechanically system comprising the hydrogel 640 and the mirror 625 can be kept stable easily. This may provide the advantage that mechanical precision of the mirror movement and as a consequence also the sensitivity of the described transducer device 600 will be enhanced.

FIG. 7 shows a drawing illustrating a swelling respectively a deswelling of a hydrogel material 740 upon a change in concentration of a monitored analyte. In a compressed state of the hydrogel material 740, analytes and analyte binding molecules, which are both connected to a backbone 742 of the hydrogel 740, are bound together with the help a free analyte. If the environmental state changes by significantly increasing the concentration of free analytes, there are enough free analytes present such that the pockets of an analyte and of a corresponding free analyte will be occupied by different free analytes. This will cause an expansion of the hydrogel material 740 to an expanded state 740a. The expansion is indicated by the arrow 741.

Accordingly, a compression of the hydrogel material 740a will be caused by a removal of the free analytes such that an analyte being coupled to a backbone 742 will directly be bound to an analyte binding molecule also being coupled to a backbone 742.

In the following there is presented a table 3, wherein examples of different hydrogels, their responsivities and a corresponding preferred transducer device configuration is given. Thereby, (a) configuration I denotes the transducer device 300 using a deflectable mirror and (b) configuration II denotes the transducer device 400 using fluorescence molecules.

The configurations I and II are complementary to each other, in a sense that configuration I is preferably suitable for hydrogels that undergo small changes (e.g. volume change <100% due to swelling) and configuration II is preferably suitable for hydrogels that undergo large changes (e.g. volume change >100% due to swelling).

TABLE 3
Examples of different hydrogels, their responsivities and
a corresponding preferred transducer device configuration.

| | | Config- |
|---|---|---|
| Hydrogel | Responsivity | uration |
| Poly-vinyl alcohol (PVA) | pH sensitive | II |
| Poly-acrylic acid (PAA) | pH sensitive | II |
| Poly-N-Isopropylacrylamides | pH sensitive | II |
| (pNIPAAm) | | |
| Carboxymethyl cellulose (CMC)/ | pH sensitive/ | II |
| Acrylic acid (AAc) | temperature sensitive | |
| Acrylonitrile (AN) copolymerized | temperature sensitive | I |
| with N-isopropylacrylamide (NPA) | | |
| (pNIPA-co-AN) | | |
| Acrylamide-maleic acid (AAM-MA) | pH sensitive | I |
| Poly-2-hydroxyethyl methacrylate | pH sensitive (4.5-7.5) | II |
| (HEMA) based | | |
| itaconic acid (IA) co-polymerized with | pH sensitive (4.5-10) | II |
| N-vinyl-2-pyrrolidone (NVP) monomer | | |
| polyelectrolyte copolymeric hydrogels | acetone, methanol, | II |
| consisting of acrylamide and itaconic | ethanol and 1-butanol | |
| acid P(AAm/IA) | sensitive | |
| pure PAAm | acetone, methanol, | II |
| | ethanol and 1-butanol | |
| | sensitive | |
| poly (2-(N,N-dimethylamino) ethyl | pH sensitive | I |
| methacrylate) | | |
| dextran-maleic anhydride (Dex-MA)/ | pH sensitive | II |
| poly(Nisopropylacrylamide) hybrid | | |
| hydrogels (dextran based in general) | | |
| Polymethacrylic acid P(MAA) | pH sensitive/salt | II |
| | concentration sensitive | |
| chitosan-g-poly(AA-co-AAm) | pH sensitive/salt | II |
| | concentration sensitive | |
| N-isopropylacrylamide, sodium | temperature sensitive | II |
| acrylate, and N-tert-butylacrylamide | | |
| based | | |
| Dex-MA/PNIPAAm hybrid hydrogels | pH sensitive/ | II |
| | temperature sensitive | |
| Hydrogels based on n-alkyl | pH sensitive | II |
| methacrylate esters (n-AMA), acrylic | | |
| acid, and acrylamide cross-linked | | |
| with 4,4-di(methacryloylamino)azo- | | |
| benzene | | |
| hydrogels based on N-t-butyl- | temperature sensitive | II |
| acrylamide (TBA), acrylamide (AAm), | | |
| 2-acrylamido-2-methylpropane | | |
| sulfonic acid sodium salt (AMPS) and | | |
| N,N0-methylenebis (acrylamide) | | |
| (BAAm) monomers | | |
| Hydrogels tailored for biosensing | Specific analyte | I |
| (see table 4) | | |

In the following there is presented a table 4, wherein examples for specifically tailored analyte binding molecules and corresponding analytes for hydrogels being suitable for biosensing are presented.

TABLE 4
Examples for analyte binding molecules and corresponding
analytes for hydrogels being suitable for biosensing.

| Analyte binding molecule | Analyte |
|---|---|
| Antibody | Antigen |
| Enzyme and Kinase | Cofactor, Substrate, Inhibitor |
| Protein A | IGG |
| Concanavalin A | D-Sugar |
| Lectins | Carbohyrates |
| Boronic acid | 1,2-cis-Diol sugars |
| Thiol | Cystein |
| Receptors (Cell membrane, | Modified molecules such as phospholated |
| cytosol, nuclear) | |
| Heparin, DNA, RNA | Protamine, Polylysine, Polyarginine |
| Poly U, Poly A, Poly Lysine, | Nucleic acid |
| Poly Arginine | |
| Triazine dye | Nucleotide |
| Commasie blue and Azure A | Arginine, Lysine, Proteins |
| Metal binding molecules | Ca ion, Mg ion, etc. |
| including chelating agents | |

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

### LIST OF REFERENCE SIGNS

100 transducer device/bio-sensitive detector/drug monitoring device
101 housing
105 radiation source/light emitting diode
106 electromagnetic radiation
120 sensor block
181 dedicated electronics
182 power source/battery
183 transmitter unit/receiver unit
200 transducer device
295 medical system
296 drug release device
298 transmission route
300 transducer device
303 recess
302 base element/electronic substrate
305 radiation source/light emitting diode
306 electromagnetic radiation
306a optical axis
325 optical element/deflectable mirror
326 electromagnetic radiation after interaction with optical element/electromagnetic radiation after reflection at deflectable mirror
340 hydrogel material
340a hydrogel material (expanded state)
341 expansion
350 radiation detector
350a detector element
352 anti reflective coating/thin film transparent layer
400 transducer device
402 base element/electronic substrate
403 recess
405 radiation source/light emitting diode/excitation light source
406 electromagnetic radiation
406a optical axis
425 optical element/fluorescence molecules
426 electromagnetic radiation after interaction with optical element/electromagnetic radiation reemitted from fluorescence molecules
440 hydrogel material
445 region of excitation
450 radiation detector
452 anti reflective coating/thin film transparent layer
500 transducer device
502 base element/electronic substrate
506 electromagnetic radiation
540 hydrogel material
540a hydrogel material (expanded state)
541 expansion
550 radiation detector
525a optical element/first semi reflective layer
525b optical element/second semi reflective layer
600 transducer device
602 base element/electronic substrate
604 second base element/second electronic substrate
606 electromagnetic radiation
625 optical element/shadowing element/movable mirror
640 hydrogel material
640a hydrogel material (expanded state)
641 expansion
650 radiation detector
740 hydrogel material
740a hydrogel material (expanded state)
741 expansion
742 backbone of hydrogel

1. A transducer device for detecting an environmental state, in particular for detecting an environmental state within a biological material, the transducer device (300, 400, 500, 600) comprising
a base element (302, 402, 502, 602),
a radiation source (305, 405), which is formed at the base element (302, 402, 502, 602) and which is adapted to emit electromagnetic radiation (306, 406),
an optical element (325, 425, 525, 625), which is arranged at the base element (302, 402, 502, 602) and which is adapted to interact with the electromagnetic radiation (306, 406) being emitted from the radiation source (305, 405),
a radiation detector (350, 450, 550, 650), which is formed at the base element (302, 402, 502, 602) and which is adapted to receive the electromagnetic radiation (326, 426) having interacted with the optical element (325, 425, 525, 625), and
a hydrogel material (340, 440, 540, 640), which is mechanically coupled to the optical element (325, 425, 525, 625) and which is adapted to change its volume when getting into contact with an environmental material of the transducer device (300, 400, 500, 600) such that the spatial position of the optical element (325, 425, 525, 625) is changed,
wherein
the base element (302, 402, 502, 602), the radiation source (305, 405) and the radiation detector (350, 450, 550, 650) are formed integrally from an electronic substrate material.

2. The transducer device according to claim 1, wherein
the optical element (325, 425, 525, 625) is formed integrally with the base element (302, 402, 502, 602).

3. The transducer device according to claim 1, further comprising
a dedicated electronic circuit arrangement (181)
for processing signals provided by the radiation detector (350, 450, 550, 650) and/or
for driving the radiation source (305, 405).

4. The transducer device according to claim 1, further comprising
a power source (182), in particular a battery (182), for providing at least the radiation source (305, 405) and the radiation detector (350, 450, 550, 650) with energy.

5. The transducer device according to claim 1, further comprising
a housing (101) having a smooth outer surface.

6. The transducer device according to claim 1, further comprising
a transmitter unit (183), which is adapted to communicate with an external receiving unit (296).

7. The transducer device according to claim 1, wherein
the radiation detector (350) has a spatial resolution, in particular the radiation detector (350) comprises an array of individual detector elements (350a).

8. The transducer device according to claim 1, wherein
the radiation detector (350, 450) is equipped with an anti-reflective coating (352, 452).

9. The transducer device according to claim 1, wherein
the optical element is a deflectable mirror (325).

10. The transducer device according to claim 1, wherein
the optical element is realized by means of fluorescence molecules (425).

11. The transducer device according to claim 10, wherein
the fluorescence molecules (425) are embedded in the hydrogel material (440).

12. The transducer device according to claim 10, wherein
the radiation source (405) is arranged relative to the radiation detector (450) in such a manner that exclusively fluorescence light (426) reaches a radiation sensitive side of the radiation detector (450).

13. The transducer device according to claim 12, wherein
the radiation sensitive side comprises a recess (403),
the radiation source (405) is located within a projection of the recess (403), and
the fluorescence molecules (425) are located within the projection of the recess (403).

14. The transducer device according to claim 1, wherein
the optical element is realized by means of a first optically semi reflective layer (525a) and a second optically semi reflective layer (525b), the two layers (525a, 525b) being oriented parallel to each other and the two layers (525a, 525b) being separated from each other by an intermediate layer (540) comprising the hydrogel material.

15. The transducer device according to claim 14, wherein
the first optically semi reflective layer (525a) is formed on a radiation sensitive side of the radiation detector (550).

16. The transducer device according to claim 1, wherein
the optical element is a shadowing element (625), which is located at least partially within the electromagnetic radiation path (606) extending from the radiation source to the radiation detector.

17. The transducer device according to claim 16, wherein
the shadowing element (625) is arranged on a radiation sensitive side of the radiation detector (650).

18. The transducer device according to claim 16, wherein
the shadowing element is a movable mirror (625).

19. A medical system comprising
the transducer device (100, 200, 300, 400, 500, 600) according to claim 1, and
a drug release device (296), which is coupled to the transducer device (100, 200, 300, 400, 500, 600) and which is adapted to release a certain amount of drug when being triggered by the transducer device (100, 200, 300, 400, 500, 600).

20. A method for detecting an environmental state, in particular for detecting an environmental state within a biological material, by means of a transducer device (300, 400, 500, 600), the method comprising the steps of
emitting electromagnetic radiation (306, 406, 506, 606) form a radiation source (305, 405), which is formed at a base element (302, 402, 502, 602) of the transducer device (300, 400, 500, 600),
directing the electromagnetic radiation (306, 406, 506, 606) to an optical element (325, 425, 525, 625), which is arranged at the base element (302, 402, 502, 602), wherein
the optical element (325, 425, 525, 625) is coupled to a hydrogel material (340, 440, 540, 640), which is adapted to change its volume when getting into contact with an environmental material of the transducer device (300, 400, 500, 600) such that the spatial position of the optical element (325, 425, 525, 625) is changed, receiving the electromagnetic radiation (326, 426), which has at least partially interacted with the electromagnetic radiation (306, 406, 506, 606) being emitted from the radiation source (305, 405) by means of a radiation detector (350, 450, 550, 650),
wherein
the base element (302, 402, 502, 602), the radiation source (305, 405) and the radiation detector (350, 450, 550, 650) are formed integrally from an electronic substrate material.
